(19) United States
(12) Patent Application Publication
(10) Pub. No.: US 2009/0187142 A1
(43) Pub. Date: Jul. 23, 2009

(54) Sleep Moisture Guard

(76) Inventor: Ana North, Joseph, OR (US)

Correspondence Address: Ana North, 102 N Lake, St/PO Box 805, Joseph, OR, 97846, US

(21) Appl. No.: 12/015,724

(22) Filed: Jan. 17, 2008

(51) Int. Cl. A61J 7/00; A61M 31/00

(52) U.S. Cl. 604/77; 604/48

(57) ABSTRACT

The mouth moisturizing device for treatment of an individual suffering from xerostomia comprises a mouth moisturizing guard, including an attaching strap to be fit around the head holding the moisture guard gently over the mouth while sleeping. A method of treatment of xerostomia and associated sleep disorders is also described

### FIELD OF THE INVENTION

My invention relates to a device providing a slow release of moisture in the mouth and to a method for treatment of xerostomia or dry mouth and associated sleep disorders in elderly individuals.

### BACKGROUND OF THE INVENTION

A dry mouth is often associated with medication, particularly heart drugs. Dry mouth is also the result of oral surgery for a malignancy, especially involving an X-Ray treatment. It may be caused by chronic sinusitis and its attendant post-nasal drip. Furthermore it is a common complaint of no known etiology of elderly individuals.

The persistence of a dry mouth at night can disturb sleep causing the individual suffering from it to waken frequently, even every hour. Furthermore peridontal disease and increased tooth decay as well as loss of teeth can be a result of xerostomia.

Current treatment has consisted of room humidifiers, and drugs, chiefly pilocarpine. Also patients with dry mouth are advised to drink water, rinse their mouth with fluids such as mouth wash, chew gum or suck hard candy. These latter suggestions of course can only be followed when the patient is awake and are impossible to perform when asleep. Room humidifiers and drugs have unfortunately proven largely ineffective.

Dry mouth is a subjective clinical symptom characterized by a less than a normal amount of saliva with no apparent boundary between normal and abnormal. Dry mouth condition occurs in all age groups and is brought about by numerous reasons, including advancing age, drug, alcohol and nicotine abuse and as side effects in the use of many commonly used prescription medicines such as anti-depressants, antihypertensives and anti-histamines among others.

### PRIOR ART

Various therapeutic measures have been recommended for patients experiencing dry mouth. Some of these include frequent rinsing with saline solutions to keep oral tissue moist and healthy. A second approach is the use of various agents to promote salivation. For example, U.S. Pat. No. 4,820,506 discloses an aqueous composition to stimulate salivation to relieve the symptoms of xerostomia wherein the composition is an acidic (pH 3-4) water solution having dissolved therein 2-3% of a food grade acidulent such as citric acid or malic acid.

Because repeated use of acidic solutions is potentially harmful to human teeth and can cause demineralization of tooth enamel, the solution is saturated with calcium phosphate to inhibit damage to tooth enamel. It has now been determined that the inhibition of demineralization afforded by the presence of saturated calcium phosphate in solutions containing citric acid is only temporary, as the calcium phosphate is supersaturated with respect to a less soluble calcium citrate which forms in the solution during storage and precipitates therefrom.

The precipitation of calcium citrate leaves the solution undersaturated with respect to calcium phosphate and the demineralization inhibition effect of the phosphate salt is as a consequence substantially diminished. Further, the presence of the calcium citrate precipitate in the solution renders the product aesthetically unappealing as well as causing malfunctioning and clogging of the spray pump systems used to apply the product to the oral cavity for relief of dry mouth condition. In addition, the acidic solution disclosed in U.S. Pat. No. 4,820,506 has been found to impart an unpleasant, pronounced sour or tart taste due to the presence of the relatively high concentration of organic acid making the product unsuitable for use as a mouth moistener.

Therefore, there is a need in the art for mouth moistener for a device which will overcome the problematic issues of sprays and lozenges of the prior art.

### OBJECT OF THE INVENTION

It is an object of my invention to provide an improved method for treating xerostomia or dry mouth which can act effectively even while the individual suffering from xerostomia sleeps without danger to the individual.

It is another object of my invention to provide an improved safe treatment for xerostomia which does not involve use of medication which is taken internally.

It is an additional object of my invention to provide a safe device which is secured over the mouth which is effective in the treatment of xerostomia even when the individual suffering from xerostomia sleeps.

It is a further object of my invention to provide a safe device for treating a sleep disorder including frequent nocturnal awakening which is the result of xerostomia

### SUMMARY OF THE INVENTION

According to my invention a method for treating an individual suffering from xerostomia comprises wearing a mouth moisturizing guard, which is of a size approximately equal to but not significantly less than the size of the cheek pouch of the individual suffering from xerostomia. Advantageously the moisture content of the mouth is retained by the moisture guard as it blocks the open mouth and stimulates the area enough for the wearer to close his or her mouth.

This treatment is completely effective providing continual moisture analogous to what occurs in an unaffected individual when the tissues of and glands in the mouth release saliva. Furthermore the treatment for xerostomia proceeds effectively during sleep so that the associated sleep disorder involving frequent awakening is alleviated. Thus an elderly individual who needs a good night sleep, particularly a heart patient, is provided with a means of getting that good sleep.

The mouth moisturizing guard according to my invention comprises a mouth guard, constructed of plastic or material of the like, and a connecting strap which holds the mouth moistening guard over the mouth. This mouth moisturizing device is particularly safe since it is not held within the mouth causing a choking and breathing problem.

The dimensions of the moisturizing guard are critical since it is to be used while an individual is sleeping. It must not be too large so that it is evident by making the individual uncomfortable. On the other hand if it were too small its effectiveness would be decreased.

### BRIEF DESCRIPTION OF THE DRAWINGS

FIG. 1

A—Attachment strap to be fit around the head holding the moisture guard gently over the mouth while sleeping.

B—Attachment point for attachment strap.

C—Mouth Moisturizing Guard made of plastic or material of the like.

FIG. 2

A—Attachment strap to be fit around the head holding the moisture guard gently over the mouth while sleeping.

B—Attachment point for attachment strap.

FIG. 3

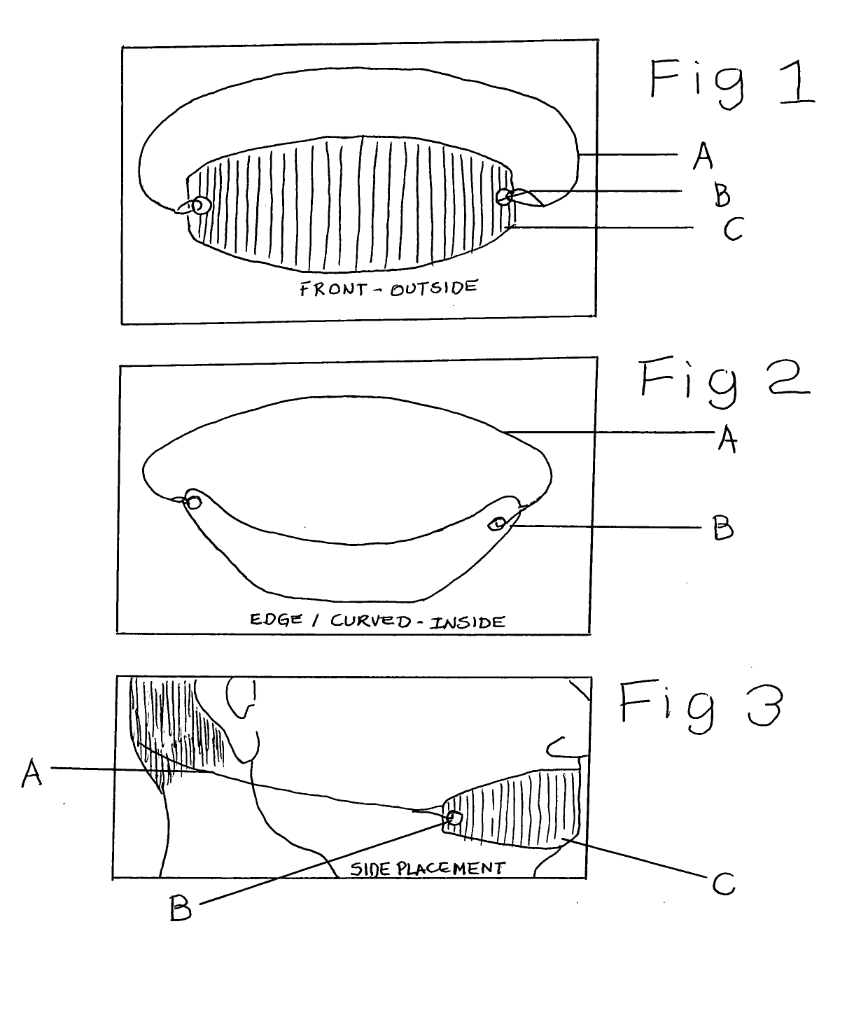

A—Attachment strap to be fit around the head holding the moisture guard gently over the mouth while sleeping.

B—Attachment point for attachment strap.

C—Mouth Moisturizing Guard made of plastic or material of the like.

While I have shown and described only two embodiments in accordance with the present invention, it is understood that the same is not limited thereto but is susceptible of numerous changes and modifications as known to those skilled in the art, and I therefore do not wish to be limited to the details shown and described herein, but intend to cover all such changes and modifications as are encompassed by the scope of the appended claims.

What is claimed is:

1. A sleep moisture guard, temporarily attached to the exterior perioral area, over both lips, supported and attached with a strap which holds the moisture guard safely in place.

2. The device of claim 1 wherein the moisture guard is manufactured of a resin based material.

3. The device of claim 1 wherein the moisture guard is manufactured of a metallic based material.

4. The device of claim 1 wherein the moisture guard is temporarily attached by an attachment strap.

5. The device of claim 1 wherein the moisture guard is embodies two attaching locations.

6. The device of claim 1 wherein the moisture guard is curved or shaped to the contour of the perioral area.
